(19) United States
(12) Patent Application Publication
(10) Pub. No.: US 2026/0176214 A1
(43) Pub. Date: Jun. 25, 2026

(54) ETHANOL TO ETHYLENE PROCESS WITH STRIPPING BYPASS

(71) Applicant: UOP LLC, Rosemont, IL (US)

(72) Inventors: Jeannie Mee Blommel, Oregon, WI (US); Kyle Krynski, Chicago, IL (US); Manuela Serban, Northbrook, IL (US); Kristen E. Allaire, Chicago, IL (US)

(21) Appl. No.: 19/380,674

(22) Filed: Nov. 5, 2025

Related U.S. Application Data
(60) Provisional application No. 63/738,224, filed on Dec. 23, 2024.

(51) Int. Cl. C07C 1/24

(52) U.S. Cl. CPC C07C 1/24

(57) ABSTRACT

A process for dehydrating ethanol to ethylene. The steps include: (a) reacting an ethanol feed stream over a dehydration catalyst to produce ethylene in a dehydration product stream; (b) separating said dehydration product stream into a product liquid stream comprising water and dissolved oxygenates and a product vapor stream of ethylene; (c) taking a bypass stream from said product liquid stream; and, (d) sending the bypass stream to the dehydration reactor bypassing a wastewater stripping column.

### FIELD

This disclosure relates to an ethanol dehydration process to produce ethylene. More particularly, this relates to a flow scheme for an ethanol dehydration process that uses water directly from a dehydration separator for process steam generators to significantly decrease the size and expense of an ethanol to jet fuel complex.

### BACKGROUND

As the world is moving fast to meet its decarbonization targets, there is a strong push to reduce carbon emissions from the aviation sector which can be achieved by using Sustainable Aviation Fuel (SAF) instead of conventional (fossil) jet fuel. SAF can be generated by many methods. One method is oligomerization of C₂-C₈ range olefins produced from a non-fossil-based ethanol, methanol or CO₂, followed by hydrogenation. The base feedstock for this route is a mixture of C₂-C₈ olefins which must have zero or minimum lifecycle CO₂ footprint for subsequent production of SAF.

Process steam is required as a non-reactive injection to the ethanol dehydration reactors to limit coke formation on the catalyst, to improve catalyst life and stability, and to help with endotherm management. Typically, process steam injection is provided in a closed-loop, non-destructive fashion with the injected process steam recovered as process water, stripped of light end impurities in a wastewater stripping column, and re-vaporized into process steam for injection to the reactors. One significant inefficiency with this current scheme is the stripping of light ends from the process water in the wastewater stripping column. Energy and equipment capital is spent to strip the light ends from the process water even though the ethanol dehydration reactors are tolerant to low levels of light end impurities. Thus, the present disclosure provides ethanol dehydration reactors that utilize process steam generated from unstripped process water. This leads to a significant decrease in the size of a wastewater stripping column and a wastewater feed bottoms exchanger and their acquisition and operating expenses.

### SUMMARY

The present disclosure describes a process for dehydrating ethanol to ethylene. The steps include: (a) reacting an ethanol feed stream over a dehydration catalyst to produce ethylene in a dehydration product stream; (b) separating said dehydration product stream into a product liquid stream comprising water and dissolved oxygenates and a product vapor stream of ethylene; and (c) taking a bypass stream from said product liquid stream.

The present disclosure also describes a process for dehydrating ethanol to ethylene. The steps include: (a) reacting the ethanol feed stream over a dehydration catalyst to produce ethylene in a dehydration product stream; (b) separating said dehydration product stream into a product liquid stream comprising water and dissolved oxygenates and a product vapor stream of ethylene; (c) taking a bypass stream from said product liquid stream; and, (d) sending the bypass stream to steam generators to generate steam to the dehydration reactor bypassing a wastewater stripping column.

The present disclosure also describes a process for dehydrating ethanol to ethylene. The steps include: (a) reacting an ethanol feed stream over a dehydration catalyst to produce ethylene in a dehydration product stream; (b) separating said dehydration product stream into a product liquid stream comprising water and dissolved oxygenates and a product vapor stream of ethylene; (c) taking a bypass stream from said product liquid stream; (d) sending the bypass stream to steam generators to generate steam to the dehydration reactor bypassing a wastewater stripping column; and, (e) taking a residual stream from said product liquid stream and stripping the residual stream in a stripping column to remove oxygenates

### BRIEF DESCRIPTION OF THE DRAWINGS

FIG. 1 provides a view of a flow scheme of a dehydration unit of an ethanol to jet complex.

While the following is described in conjunction with specific embodiments, it will be understood that this description is intended to illustrate and not limit the scope of the preceding description and the appended claims.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure. As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “Cx” is to be understood to refer to molecules having the number of carbon atoms represented by the variable, “x”. Similarly, the term “Cx−” refers to molecules that contain less than or equal to x and preferably x and less carbon atoms. The term “Cx+” refers to molecules with more than or equal to x and preferably x and more carbon atoms.

### DETAILED DESCRPTION

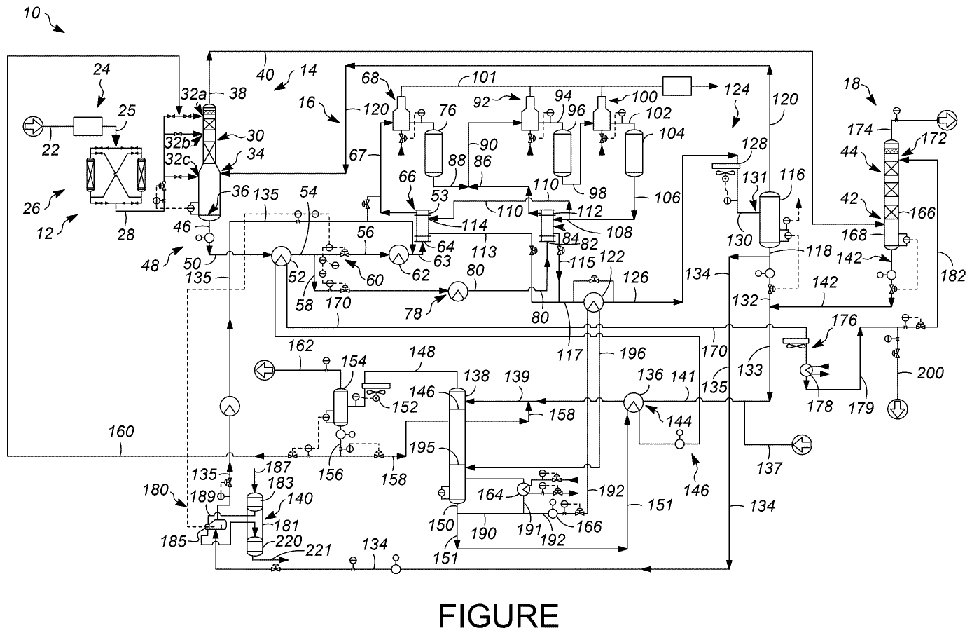

FIG. 1 shows a flow scheme of a dehydration unit 10 divided into four main sections, an ethanol feed pretreatment section 12, oxygenate (diethyl ether) absorber section 14, a reactor section 16, and a water wash section 18. In the ethanol feed pretreatment section 12, an incoming fresh ethanol stream 22 is charged to a feed filter 24 to remove particulate materials and then through line 25 to an ion-exchange resin guard bed 26 where metals contaminants, and basic components are removed. The ion-exchange resin guard beds 26 may be configured in a lead/lag flow scheme such that one vessel can be taken offline and regenerated while one vessel is online.

The treated ethanol stream in line 28 exiting the resin guard beds 26 is charged to a diethyl ether (“DEE”) absorber column 30 through an inlet 32. The DEE absorber column 30 is suffused with a dehydration separator vapor stream which enters through line 120 below the bottom of the packing at an inlet 34. The DEE absorber column 30 is provided to remove diethyl ether from the dehydration separator vapor stream by contacting the treated ethanol stream in line 28 with the dehydration separator vapor stream in line 120. The treated ethanol stream absorbs DEE from the dehydration separator vapor stream to recycle it to the dehydration reaction. A DEE absorber bottom sump is designed to provide about 1 to about 30 minutes residence time to the liquid feed entering the reactor section 16. A top stream in line 38 of the DEE absorber column 30 contains ethylene product depleted of DEE and is charged in a stream through line 40 to an inlet 42 of a wash water tower 44. The treated ethanol stream in line 28 is fed to the DEE absorber column through one or all of three inlets 32a-32c. The bottom inlet 32c is utilized to feed the treated ethanol stream until the dehydration catalyst begins to deactivate. The intermediate inlet 32b and then the top inlet 32c are then used to feed the ethylene stream to the DEE absorber column 30 as the dehydration catalyst becomes more spent. The recycle stream 160 can also be mixed with the treated ethanol feed stream at the inlet being utilized by management of the valves.

The DEE absorber bottoms liquid stream in line 46 of ethanol as a principal component is pumped to the reactor section 16 via a dehydration charge pump 48 through line 50 to an ethanol treated water heat exchanger 52 where it is preheated by heat exchange with a treated water stream in line 170. The preheated ethanol stream in line 54 is split into two streams in lines 56 and 58 at split 60. The first split stream in line 56 is heated and vaporized in a first ethanol stream heater 62, and the vaporized stream is mixed with the steam generated from the bypass product liquid stream from the bottom of a dehydration product separator 116 in line 135. The combined stream is conveyed through line 63 into a cold side (tube side) 64 of a first combined feed exchanger (CFE1) 66 to be heat exchanged with a third reactor effluent stream in line 110. A first heated split stream is then transported through line 67 to a charge heater 68. Some of the bypass product liquid stream in line 135 can be bypassed around the first CFE1 66 to a line 67. The first heated split stream in line 67 is heated to required reaction temperature in the charge heater 68 and routed to the first reactor 76.

The second split stream in line 58 is heated and vaporized in heat exchanger 78 perhaps by heat recovery in the oligomerization section 180 and conveyed through line 80 to the cold side (tube side) 82 of a second combined feed exchanger (CFE2) 84 to be heat exchanged with a third reactor effluent stream in line 108. From a cold side outlet from CFE2 84, the heated feed stream in line 86 is mixed with a first reactor effluent 88 and routed through line 90 to a first interheater 92 where the stream is further heated to a required reaction temperature before being conveyed through line 94 to a second reactor 96. The ethanol feed is converted to ethylene and water over a dehydration catalyst at a pressure of about 210 kPa (gauge) (30 psig) to about 1000 kPa (gauge) (145 psig). Steam does not take part in the reaction (except maybe some minor side reactions) but the steam added in the reactor serves the dual purpose of controlling the endotherm across the reactor as well as maintaining the stability of the catalyst (reducing coke laydown). Minimizing temperature drop across the second reactor 96 is critical because at the lower reactor outlet temperature, the formation of diethyl ether is more pronounced. To ensure that the byproducts formation is limited, a second reactor effluent 98 is passed through a second interheater 100 and again heated up to required reactor temperature before routing through line 102 to a third reactor 104. The fired heaters 68, 92 and 100 used in the reactor section 16 are designed as natural draft furnaces with the main process heating occurring in the radiant section while the convection section of these fired heaters is designed to generate high pressure steam. A combined vapor stream 101 from the interheaters is collected as high-pressure steam.

The third reactor 104 is a polishing reactor which ensures that the byproducts along with unconverted ethanol is converted to useful ethylene. A third reactor effluent 106 is split into two streams in line 108 and 110. The third reactor effluent stream in line 108 passes through the hot side (shell side) 112 of CFE2 84 and the stream in line 110 passes through the hot side (shell side) 114 of CFE1 66. The cooled third reactor effluent streams from the side outlets in lines 113, 115 respectively from CFE1 66 and CFE2 84, combine to form stream in line 117 which is cooled in the wastewater stripper reboiler 122 by heat exchange with a wastewater stripper bottoms stream in line 192 and conveyed through line 126 to an inlet 128 of the dehydration product condenser 124 and then through line 130 to an inlet 131 of the dehydration product separator 116.

In the dehydration product separator 116, the dehydration product is separated into an overhead product vapor stream in line 120 and bottoms product liquid stream in line 118. The dehydration product separator liquid stream in line 118 is predominantly water with some dissolved oxygenates and the vapor stream in line 120 is essentially the ethylene product. The dehydration separator vapor stream in line 120 is routed to the DEE absorber column 30.

The water wash section 18 comprises the wastewater stripping column 138 and the water wash tower 44. The dehydration product liquid stream in bottoms line 118 from the dehydration product separator 116 may be split into a first product liquid stream in line 132 and a second bypass product liquid stream in line 134. The first product liquid stream in line 132 is combined with the water wash tower bottoms in line 142 to make a wastewater stream in line 133. The wastewater stream in line 133 is supplemented with a wastewater stream in line 137 to provide a supplemented wastewater stream in line 141. The supplemented wastewater stream in line 141 is routed through the shell side 144 of the wastewater stripper feed bottoms exchanger 136 to be heat exchanged with the water stripper bottoms in line 151. The heated supplemented wastewater stream in line 141 is then mixed with the stripper reflux stream in line 158, the effluent from an off-gas knockout drum 154, to provide a wastewater feed stream in line 139 which is fed to a low-pressure wastewater stripping column 138. The wastewater feed stream enters a top tray 146 of the wastewater stripping column 138.

The second bypass product liquid stream in line 134 is routed to the oligomerization and hydrogenation sections to generate steam and returns to mix with the effluent from the ethanol steam heater 62 to form the mixed stream 63. The second bypass product liquid stream in line 134 is not directed through the wastewater stripper feed bottoms exchanger 136 and the wastewater stripping column 138 but bypasses the stripping operation and for that matter the entire water wash section 18.

The wastewater stripping column 138 is designed to strip off the unconverted oxygenates in the feed as an overhead vapor product stream 148 while recovering treated water in the bottoms line 150. The wastewater stripping column 138 operates at about 5 psig (35 kPa) to about 10 psig (70 kPa). The overhead vapor in the overhead line 148 is cooled and condensed in an off-gas condenser 152 before entering an off-gas knockout drum 154. The off-gas knockout drum liquid stream in line 156 has a predominance of the alcohols carried over from the DEE absorber vapor stream, unconverted alcohols from the reactor, water along with other non-selective oxygenates formed within the reactor such as acetaldehyde, ethers, acetic acid etc. The off-gas knockout drum liquid in line 156 is split between a reflux stream in line 158 which is recycled and mixed with the wastewater stream in line 133 feeding to the wastewater stripping column 138 and a recycle stream routed through line 160 to the DEE absorber column 30. The off-gas knockout drum vapor stream in line 162 is a small purge stream which is a mix of olefins, dissolved in the dehydration separator and water wash tower liquid, as well as oxygenates.

The wastewater stripping column 138 has a two-reboiler system. The wastewater stripper auxiliary reboiler 164 utilizes low pressure steam as reboiling medium, while the wastewater stripper reboiler 122 is process heat integrated with the hot dehydration reactor effluent upstream of the dehydration product condenser. The wastewater stripper bottoms 150 is split into two streams in lines 190 and 151. The stream in line 190 splits into lines 191 and 192. The stream in line 191 is heated in the auxiliary wastewater stripper reboiler 164 and returned to the wastewater stripping column 138 below a bottom deck 195. The stream in line 192 is pumped by treated water pumps 166 and directed to the wastewater stripper reboiler 122 to be heated by heat exchange with the third reactor combined effluent stream from CFE1 66 and CFE2 84 in line 117 and returned in a stream through line 196 back to the wastewater stripping column 138 below the bottom deck 195. The stream in line 151 is the treated water used for vapor product oxygenate wash in the water wash tower 44. The stripper bottom stream in line 151 is heat exchanged with the supplemented wastewater stream in line 141 in the wastewater stripper feed bottoms heat exchanger 136 and then heat exchanged with the DEE absorber bottom stream in line 50 in the ethanol-treated water exchanger 52 to give over its heat and provide a cooled treated water stream in line 170. The cooled treated water stream in line 170 is further cooled in a treated water cooler 176 and then in a treated water trim cooler 178. The treated water stream in line 179 splits into two streams in lines 182 and 200. The stream in line 182 is fed into the water wash tower 44. The stream in line 200 primarily contains water and is captured as a product.

As noted previously, the dehydration separator vapor stream in line 120 can be routed to the DEE absorber 30. The dehydration separator vapor stream in line 120 has certain oxygenates such as acetaldehyde, diethyl ether, dimethyl ether, water, unconverted alcohols etc. which may need to be removed, depending on the further use of the ethylene product.

In the DEE absorber column 30, the diethyl ether in the separator vapor in line 120 is absorbed in the liquid in bottoms line 46 along with some other oxygenates. The DEE absorber column 30 may comprise beds of packing such as Raschig rings. Trays or structural packing are also contemplated. Since an ethanol stream 28 is used for washing the separator vapor stream in line 120, there is carry over of some ethanol feed to the DEE absorber vapors in line 38. Therefore, the DEE absorber overhead vapors in line 38 are routed to below the bottom bed 166 of the water wash tower 44. The water wash tower 44 is designed to wash off oxygenates such as acetaldehyde, unconverted alcohols from the reactor section, ethanol carryover from the DEE absorber vapors, acetic acid etc. from ethylene product using treated water from the wastewater stripper bottoms 150. The treated water stream in line 182 from the ethanol treated water exchanger 52 enters the top bed 172 of the water wash tower 44 and absorbs oxygenates in a counter-current direction over multiple beds. The water wash tower overhead ethylene vapor in the wash overhead line 174 are collected, dried, compressed, and further fractionated prior to routing to the oligomerization section 180, while the liquid bottoms stream in line 142 with all the dissolved oxygenates including alcohols are routed to the wastewater stripping column 138 along with the first product liquid stream in line 132.

The oligomerization zone 180 has an oligomerization reactor 181 with two catalyst beds 183 and a steam generator(s) 185. A light olefin stream 187 of C2-C8 olefins is oligomerized into higher olefins and exits the first catalyst bed 183 in line 189. The oligomerized effluent stream in line 189 is heat exchanged with the bypass product liquid stream in line 134 in steam generator(s) 185. The bypass product liquid stream in line 134 absorbs heat from the oligomerized effluent by indirect heat exchange to vaporize the bypass product liquid stream to steam which exits as a product steam stream in line 135. The cooled oligomerized effluent stream in line 189 is oligomerized in a second catalyst bed 220 and exits the oligomerization reactor in 181 in line 221. The light olefin stream 187 may be at a temperature of about 20° C. (68° F.) to about 150° C. (302° F.) and a pressure of about 2.16 MPag (350 psig), preferably about 6.2 MPag (900 psig), to about 8.4 MPag (1200 psig).

Suitable oligomerization catalyst may include a zeolitic catalyst. The oligomerization catalyst may be considered a solid acid catalyst. The zeolite may comprise between about 5 and about 95 wt % of the catalyst, for example between about 5 and about 85 wt %. Suitable zeolites include zeolites having a structure from one of the following classes: MFI, MEL, ITH, IMF, TUN, FER, BEA, FAU, BPH, MEI, MSE, MWW, UZM-8, MOR, OFF, MTW, TON, MTT, AFO, ATO, and AEL. Three-letter codes indicating a zeotype are as defined by the Structure Commission of the International Zeolite Association and are maintained at http://www.iza-structure.org/databases. UZM-8 is as described in U.S. Pat. No. 6,756,030. In a preferred aspect, the oligomerization catalyst may comprise a zeolite with a framework having a ten-ring pore structure. Examples of suitable zeolites having a ten-ring pore structure include TON, MTT, MFI, MEL, AFO, AEL, EUO and FER. In a further preferred aspect, the oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include MTT. In a further aspect, the oligomerization catalyst comprises an MTT zeolite.

### EXAMPLE

In a simulation of a dehydration reactor unit that converts ethanol to ethylene in the process disclosed herein in which the liquid stream from the dehydration product separator 116 in line 134 bypasses the wastewater stripper feed bottoms exchanger 136 and the wastewater stripping column 138, the liquid bypass stream enabled a reduction in surface area of the wastewater stripper feed bottoms exchanger 136 by about 23%. Additionally, a diameter of the wastewater stripping column 138 decreased by about 21% and passes per tray was cut in half. Also, both fuel gas and electrical utility usages decreased.

### SPECIFIC EMBODIMENTS

While the following is described in conjunction with specific embodiments, it will be understood that this description is intended to illustrate and not limit the scope of the preceding description and the appended claims.

A first embodiment of the disclosure is a process for dehydrating ethanol to ethylene comprising reacting an ethanol feed stream over a dehydration catalyst to produce ethylene in a dehydration product stream; separating the dehydration product stream into a product liquid stream comprising water and dissolved oxygenates and a product vapor stream of ethylene; and taking a bypass stream from the product liquid stream. An embodiment of the disclosure is one, any or all of prior embodiments in this paragraph up through the first embodiment in this paragraph further comprising sending the bypass stream to a dehydration reactor bypassing a wastewater stripping column. An embodiment of the disclosure is one, any or all of prior embodiments in this paragraph up through the first embodiment in this paragraph further comprising taking a residual stream from the product liquid stream and stripping the residual stream in a stripping column to remove oxygenates. An embodiment of the disclosure is one, any or all of prior embodiments in this paragraph up through the first embodiment in this paragraph wherein the step of providing an ethanol stream further comprises removing metals from the filtered ethanol stream with an ethanol treater column. An embodiment of the disclosure is one, any or all of prior embodiments in this paragraph up through the first embodiment in this paragraph further comprising contacting an ethanol stream with the product vapor stream to absorb diethyl ether from the product vapor stream into the ethanol stream to provide the ethanol feed stream and an absorbed product stream. An embodiment of the disclosure is one, any or all of prior embodiments in this paragraph up through the first embodiment in this paragraph further comprising washing the absorbed product stream with a water stream to wash oxygenates from the absorbed product stream into the water stream. An embodiment of the disclosure is one, any or all of prior embodiments in this paragraph up through the first embodiment in this paragraph further comprising stripping the water stream with washed oxygenates in a wastewater stripping column to remove oxygenates. An embodiment of the disclosure is one, any or all of prior embodiments in this paragraph up through the first embodiment in this paragraph further comprising vaporizing the bypass stream to steam before sending it to the dehydration reactor. An embodiment of the disclosure is one, any or all of prior embodiments in this paragraph up through the first embodiment in this paragraph further comprising heating the bypass stream by indirect heat exchange with oligomerization effluent and other hot product streams.

A second embodiment of the disclosure is a process for dehydrating ethanol to ethylene comprising reacting the ethanol feed stream over a dehydration catalyst to produce ethylene in a dehydration product stream; separating the dehydration product stream into a product liquid stream comprising water and dissolved oxygenates and a product vapor stream of ethylene; taking a bypass stream from the product liquid stream; and sending the bypass stream to the dehydration reactor bypassing a wastewater stripping column. An embodiment of the disclosure is one, any or all of prior embodiments in this paragraph up through the second embodiment in this paragraph further comprising taking a residual stream from the product liquid stream and stripping the residual stream in a stripping column to remove oxygenates. An embodiment of the disclosure is one, any or all of prior embodiments in this paragraph up through the second embodiment in this paragraph wherein the step of providing an ethanol stream further comprises removing metals from the filtered ethanol stream with an ethanol treater column. An embodiment of the disclosure is one, any or all of prior embodiments in this paragraph up through the second embodiment in this paragraph wherein the step of removing diethyl ether further comprises contacting an ethanol stream with the product vapor stream to absorb diethyl ether from the product vapor stream into the ethanol stream to provide the ethanol feed stream and an absorbed product stream. An embodiment of the disclosure is one, any or all of prior embodiments in this paragraph up through the second embodiment in this paragraph further comprising washing the absorbed product stream with a water stream to wash oxygenates from the absorbed product stream into the water stream. An embodiment of the disclosure is one, any or all of prior embodiments in this paragraph up through the second embodiment in this paragraph further comprising stripping the water stream with washed oxygenates in a wastewater stripping column to remove oxygenates. An embodiment of the disclosure is one, any or all of prior embodiments in this paragraph up through the second embodiment in this paragraph further comprising vaporizing the bypass stream to steam before sending it to the dehydration reactor. An embodiment of the disclosure is one, any or all of prior embodiments in this paragraph up through the second embodiment in this paragraph further comprising heating the bypass stream by indirect heat exchange with oligomerization effluent and other hot product streams.

A third embodiment of the disclosure is a process for dehydrating ethanol to ethylene comprising reacting the ethanol feed stream over a dehydration catalyst to produce ethylene in a dehydration product stream; separating the dehydration product stream into a product liquid stream comprising water and dissolved oxygenates and a product vapor stream of ethylene; taking a bypass stream from the product liquid stream; sending the bypass stream to the dehydration reactor bypassing a wastewater stripping column; and taking a residual stream from the product liquid stream and stripping the residual stream in a stripping column to remove oxygenates. An embodiment of the disclosure is one, any or all of prior embodiments in this paragraph up through the third embodiment in this paragraph further comprising contacting an ethanol stream with the product vapor stream to absorb diethyl ether from the product vapor stream into the ethanol stream to provide the ethanol feed stream and an absorbed product stream. An embodiment of the disclosure is one, any or all of prior embodiments in this paragraph up through the third embodiment in this paragraph further comprising washing the absorbed product stream with a water stream to wash oxygenates from the absorbed product stream into the water stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for dehydrating ethanol to ethylene comprising:
reacting an ethanol feed stream over a dehydration catalyst to produce ethylene in a dehydration product stream;
separating said dehydration product stream into a product liquid stream comprising water and dissolved oxygenates and a product vapor stream of ethylene; and
taking a bypass stream from said product liquid stream.

2. The process of claim 1 further comprising sending the bypass stream to steam generators to generate steam to a dehydration reactor bypassing a wastewater stripping column.

3. The process of claim 1 further comprising taking a residual stream from said product liquid stream and stripping the residual stream in a stripping column to remove oxygenates.

4. The process of claim 1 wherein the step of providing an ethanol stream further comprises removing metals from the filtered ethanol stream with an ethanol treater column.

5. The process of claim 4 further comprising contacting an ethanol stream with the product vapor stream to absorb diethyl ether from said product vapor stream into said ethanol stream to provide said ethanol feed stream and an absorbed product stream.

6. The process of claim 5 further comprising washing said absorbed product stream with a water stream to wash oxygenates from said absorbed product stream into the water stream.

7. The process of claim 6 further comprising stripping the water stream with washed oxygenates in a wastewater stripping column to remove oxygenates.

8. The process of claim 1 further comprising vaporizing said bypass stream to steam before sending it to the dehydration reactor.

9. The process of claim 8 further comprising heating said bypass stream by indirect heat exchange with oligomerization effluent and other hot product streams.

10. A process for dehydrating ethanol to ethylene comprising:
reacting the ethanol feed stream over a dehydration catalyst to produce ethylene in a dehydration product stream;
separating said dehydration product stream into a product liquid stream comprising water and dissolved oxygenates and a product vapor stream of ethylene;
taking a bypass stream from said product liquid stream; and
sending the bypass stream to steam generators to generate steam to the dehydration reactor bypassing a wastewater stripping column.

11. The process of claim 10 further comprising taking a residual stream from said product liquid stream and stripping the residual stream in a stripping column to remove oxygenates.

12. The process of claim 10 wherein the step of providing an ethanol stream further comprises removing metals from the filtered ethanol stream with an ethanol treater column.

13. The process of claim 10 wherein the step of removing diethyl ether further comprises contacting an ethanol stream with the product vapor stream to absorb diethyl ether from said product vapor stream into said ethanol stream to provide said ethanol feed stream and an absorbed product stream.

14. The process of claim 13 further comprising washing said absorbed product stream with a water stream to wash oxygenates from said absorbed product stream into the water stream.

15. The process of claim 14 further comprising stripping the water stream with washed oxygenates in a wastewater stripping column to remove oxygenates.

16. The process of claim 10 further comprising vaporizing said bypass stream to steam before sending it to the dehydration reactor.

17. The process of claim 16 further comprising heating said bypass stream by indirect heat exchange with oligomerization effluent and other hot product streams.

18. A process for dehydrating ethanol to ethylene comprising:
reacting the ethanol feed stream over a dehydration catalyst to produce ethylene in a dehydration product stream;
separating said dehydration product stream into a product liquid stream comprising water and dissolved oxygenates and a product vapor stream of ethylene;
taking a bypass stream from said product liquid stream;
sending the bypass stream to steam generators to generate steam to the dehydration reactor bypassing a wastewater stripping column; and
taking a residual stream from said product liquid stream and stripping the residual stream in a stripping column to remove oxygenates.

19. The process of claim 18 further comprising contacting an ethanol stream with the product vapor stream to absorb diethyl ether from said product vapor stream into said ethanol stream to provide said ethanol feed stream and an absorbed product stream.

20. The process of claim 19 further comprising washing said absorbed product stream with a water stream to wash oxygenates from said absorbed product stream into the water stream.
